(19) United States
(12) Patent Application Publication
(10) Pub. No.: US 2025/0066757 A1
(43) Pub. Date: Feb. 27, 2025

(54) A METHOD FOR SEPARATING SUPERCOILED PLASMID DNA

(71) Applicant: Cytiva BioProcess R&D AB, Uppsala (SE)

(72) Inventors: Simon Aberg, Uppsala (SE); Linda Hagman, Uppsala (SE); Mikael Berg, Uppsala (SE); Hans Blom, Uppsala (SE); Albin Larsson, Uppsala (SE); Joakim Bagge, Uppsala (SE); Elin Monie, Uppsala (SE); Markus Pitkanen, Uppsala (SE)

(21) Appl. No.: 18/719,120

(22) PCT Filed: Feb. 20, 2023
(86) PCT No.: PCT/EP2023/054159

§ 371 Date: Jun. 12, 2024

(30) Foreign Application Priority Data
Mar. 1, 2022 (GB) 2202777.5

(51) Int. Cl. C12N 15/10

(52) U.S. Cl. CPC C12N 15/101; C12N 15/1017

(57) ABSTRACT

The present disclosure is directed to a method for separating supercoiled plasmid DNA (pDNA) from a liquid sample. the method comprising the steps of: (a) adding a liquid sample comprising pDNA to a first chromatography material comprising (i) an anion exchange chromatography ligand for binding to pDNA and (ii) a support material allowing convective flow through the first chromatography material, wherein the liquid sample originates from a cell culture harvest and has been subjected to a step of removing RNA before step (a): (b) eluting a liquid sample. comprising a purified mixture of supercoiled pDNA and open circular pDNA, from the first chromatography material: (c) adding the liquid sample from step (b) to a second chromatography material comprising a ligand that binds to pDNA and enables selective separation of supercoiled pDNA from open circular pDNA: (d) eluting the purified supercoiled pDNA from the second chromatography material: wherein the supercoiled pDNA eluted in step (d) has a purity degree of at least 95% without use of any further chromatography material than said first and second chromatography materials. Steps (a)-(d) and any intermediate steps can be completed within 5 hours. Further disclosed are uses of supercoiled pDNA obtained by said separation method.

### FIELD OF THE DISCLOSURE

The present disclosure relates to the field of separation of plasmid DNA (pDNA) and is directed to a method for producing a purified composition comprising pDNA, as well as a method for separating supercoiled pDNA from a cell culture harvest. Further disclosed are uses of supercoiled pDNA obtained by said method.

### BACKGROUND OF THE DISCLOSURE

Plasmids are small DNA molecules that are physically separated from chromosomal DNA. They are small, circular, double-stranded DNA molecules that replicate independently in bacteria.

Plasmid DNA (pDNA) is an important genetic engineering tool used to clone and amplify or express genes in genetic and biotechnology labs. Production of viral vectors and mRNA is dependent on pDNA. It can be used as a template for mRNA manufacturing and can be used for manufacturing viral vectors to produce vector-based DNA vaccines or applied for cell and gene therapy. Good manufacturing practices (GMP)-grade pDNA can be used directly as DNA vaccines, in gene therapy, and in ex vivo applications, e.g. in recombinant protein production.

Plasmid supply and quality is of key importance for manufacturing of viral vectors, mRNA, and DNA vaccines. Processes for GMP manufacturing of pDNA must be designed to meet output and quality needs. Different plasmids and applications (such as different therapies) have different requirements. The pDNA needs to be expressed and purified in high yields and titers, meeting the yield and purity goals of each application.

Supercoiled (SC) plasmids in the range of 5 to 20 kb pairs (kbp) are the most common in bioprocess applications. Previously known processes for the purification of supercoiled plasmids are described for example in Lemmens et al (2003), Sandberg et al (2004), and Hitchcock et al (2010). Further, Application Note 28-4094-85 AA (Cytiva, Sweden) proposes a 3-step method involving 1) group separation on a size exclusion chromatography resin, 2) capture and selective desorption of SC pDNA, and 3) polishing using a rigid bead chromatography resin with a strong anion exchange ligand.

However, there is always a need for novel purification strategies to increase e.g. the productivity and sustainability of large-scale methods for purification of pDNA.

### SUMMARY OF THE DISCLOSURE

The present disclosure aims to overcome or at least partly alleviate drawbacks of the prior art.

Accordingly, it is an object of the present disclosure is to provide a more productive method for separating supercoiled pDNA.

In one aspect, the present disclosure provides a method for separating supercoiled plasmid DNA (pDNA) from a liquid sample, the method comprising the steps of:
(a) adding a liquid sample comprising pDNA to a first chromatography material comprising (i) an anion exchange chromatography ligand for binding to pDNA and (ii) a support material allowing convective flow through the first chromatography material, wherein the liquid sample originates from a cell culture harvest and has been subjected to a step of removing RNA before step (a);
(b) eluting a liquid sample, comprising a purified mixture of supercoiled pDNA and open circular pDNA, from the first chromatography material;
(c) adding the liquid sample from step (b) to a second chromatography material comprising a ligand that binds to pDNA and enables selective separation of supercoiled pDNA from open circular pDNA;
(d) eluting the purified supercoiled pDNA from the second chromatography material;

wherein the supercoiled pDNA eluted in step (d) has a purity degree of at least 95% without use of any further chromatography material than said first and second chromatography materials.

Step (a) may be preceded by a step (a1) of pre-treating the liquid sample, wherein said pre-treating comprises at least one of: subjecting the cell culture harvest to cell lysis, precipitation of RNA in the presence of an RNA-precipitating agent, clarification, and filtration.

Subjecting the cell culture harvest to cell lysis may be performed by mixing the cell culture harvest with a lysis solution using a static mixer. The cell culture harvest may be contacted with said lysis solution to form a combined composition, and said combined composition is then allowed to flow through said static mixer to achieve mixing thereof.

Said step (a1) may provide said step of RNA removal, by precipitation of RNA in the presence of an RNA-precipitating agent. The RNA-precipitating agent may be selected from the group consisting of calcium chloride, lithium chloride, and tripotassium citrate, and is preferably preferably calcium chloride.

In another aspect, the present disclosure provides method for producing a composition comprising pDNA, comprising
(I) providing a cell culture harvest in the form of a slurry comprising pDNA producer cells;
(II) contacting said cell culture harvest with a lysis solution to form a lysis composition;
(III) mixing the lysis compositing using a static mixer to form a cell lysate;
(IV) adding a neutralization solution to the cell lysate;
(V) optionally precipitating RNA in the cell lysate by action of an RNA-precipitating agent;
(VI) clarifying the cell lysate;
(VII) filtering the cell lysate to obtain a solution comprising plasmid DNA (pDNA)
(VIII) subjecting said solution comprising pDNA to at least one chromatography step to provide a purified composition comprising pDNA.

The steps (I) to (VIII) may be performed continuously, using single use equipment. The process is fast and can be readily controlled.

The at least one chromatography step may comprise at least one bind-elute chromatography step to capture pDNA, and may in particular comprise chromatography steps (a)-(d) as described herein.

The methods disclosed herein typically do not comprise a size-exclusion chromatography step, such as group separation. Hence, the methods according to the first or the second aspect can be performed without the use of a size exclusion chromatography resin.

In contrast to size exclusion chromatography, step (a) of the present process provides high loading capacity and significantly contributes to shortening of the overall process time for a given volume to be processed. For example, steps (a)-(d) and any intermediate steps can be completed within 5 hours. Further, as demonstrated in the examples, a substantial reduction in ammonium sulfate consumption can be achieved. The process meets industry acceptance criteria of GMP grade pDNA.

Hence, the 2-step chromatography method disclosed herein provides supercoiled pDNA of high purity and allows a higher productivity. In addition, the present method also reduces the buffer consumption compared to previous methods, thus providing a more sustainable process for pDNA separation.

The above-disclosed methods may further comprise subjecting the supercoiled pDNA eluted in step
(d) to one or more of the following steps:
(e1) concentrating the supercoiled pDNA to a pharmaceutically relevant dose,
(e2) replacing a buffer applied in step (d) of the above-described method with a pharmaceutically acceptable buffer, and/or
(e3) sterilizing the supercoiled pDNA,
   thereby obtaining a composition comprising pharmaceutical grade supercoiled pDNA, optionally wherein the composition is a pharmaceutical composition or an active pharmaceutical ingredient comprising pharmaceutical grade supercoiled pDNA.

The present disclosure further provides a method for preventing or treating a disease or disorder in a subject, comprising administering to the subject a pharmaceutical composition or an active pharmaceutical ingredient comprising pharmaceutical grade supercoiled pDNA obtained by performing the herein disclosed separation method, optionally wherein the active pharmaceutical ingredient is administered in the form of a DNA vaccine, and the disease or disorder to be prevented is caused by an infection by a virus, such as SARS-COV-2, Zika, Influenza A, yellow fever virus, or human immunodeficiency virus (HIV).

In another aspect, the present disclosure is directed to a method for producing a viral vector, comprising transfecting cells in vitro with a composition comprising pharmaceutical grade supercoiled pDNA obtained by performing the herein disclosed separation method, thereby enabling production of a viral vector, optionally wherein the viral vector is for use as an active pharmaceutical ingredient.

In a further aspect, the present disclosure also provides a method for producing RNA, comprising using the composition comprising pharmaceutical grade supercoiled pDNA obtained by performing the herein disclosed separation method, as a template to produce RNA, optionally wherein the RNA is for use as an active pharmaceutical ingredient.

Preferred aspects of the present disclosure are described below in the detailed description and in the dependent claims. It is noted that the invention relates to all possible combinations of features recited in the claims.

### BRIEF DESCRIPTION OF THE DRAWINGS

FIG. 1 is a flow chart of a method for separating supercoiled pDNA, comprising steps (a)-(d), according to the present disclosure.

FIG. 2 is a flow chart of the method for separating supercoiled pDNA according to FIG. 1, further comprising an additional step (a1) before step (a).

FIG. 3 is a flow chart of the method for separating supercoiled pDNA according to FIG. 1, optionally comprising step (a1) before step (a), further comprising one or more additional steps (e1), (e2) and/or (e3) after step (d).

FIG. 4 is a photograph of an agarose gel showing RNA reduction by CaCl₂ precipitation during cell lysis.

FIG. 5 is a graph showing the elution curves for three cycles of separation of pDNA according to the first chromatography step of the separation method as described in the Example below.

FIG. 6 is a graph showing the elution curve for separation of supercoiled pDNA and open circular pDNA according to the second chromatography step of the separation method as described in the Example below.

### DETAILED DESCRIPTION OF THE DISCLOSURE

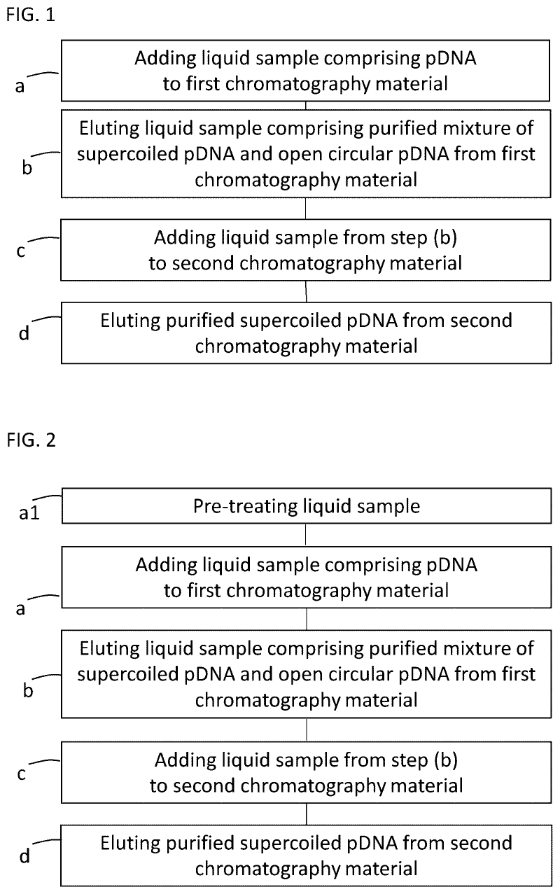

The present disclosure solves or at least mitigates the problems associated with existing methods for separating supercoiled plasmid DNA (pDNA) by providing, as illustrated in FIG. 1, a method for separating supercoiled pDNA from a liquid sample, the method comprising the steps of:
(a) adding a liquid sample comprising pDNA to a first chromatography material comprising (i) an anion exchange chromatography ligand for binding to pDNA and (ii) a support material allowing convective flow through the first chromatography material, wherein the liquid sample originates from a cell culture harvest and has been subjected to a step of removing RNA before step (a);
(b) eluting a liquid sample, comprising a purified mixture of supercoiled pDNA and open circular pDNA, from the first chromatography material;
(c) adding the liquid sample from step (b) to a second chromatography material comprising a ligand that binds to pDNA and enables selective separation of supercoiled pDNA from open circular pDNA;
(d) eluting the purified supercoiled pDNA from the second chromatography material; wherein the supercoiled pDNA eluted in step (d) has a purity degree of at least 95% without use of any further chromatography material than said first and second chromatography materials.

Significant advantages of the presently disclosed method, containing only two chromatography steps, include obtaining supercoiled pDNA of high purity (at least 95%) at an 80% reduction in production time for downstream processing and a 50% reduction in ammonium sulfate consumption, thereby resulting in increased sustainability, as compared to the previously known 3-step method for pDNA purification described in Application Note 28-4094-85 AA (Cytiva, Sweden).

It is to be understood that the term “liquid sample” as used herein encompasses any type of sample obtainable from a cell culture, or from a fluid originating from a cell culture which fluid is at least partly purified, by any means of separation and purification.

The term “supercoiled pDNA” refers to plasmid DNA in the form of a closed loop which has a coiled topology. DNA may be positively or negatively supercoiled. For example, supercoiled plasmid DNA may form a two-start right-handed helix with terminal loops.

The term “open circular pDNA” refers to plasmid DNA in which one of the strands of the DNA double helix has been broken, with the effect that at least part of the strain on the double helix, that causes plasmids to assume a supercoiled configuration, has been released. Thereby, open circular pDNA forms a closed loop which is not coiled, but maintains a circular topology.

The term “purity” with regard to supercoiled plasmid DNA refers to the content of supercoiled plasmid DNA in relation to the total amount of plasmid DNA. “Plasmid DNA” generally includes supercoiled plasmid DNA, open circular plasmid DNA as linear plasmid DNA, if present. Accordingly, supercoiled pDNA of a purity of 95% means that the supercoiled pDNA constitutes 95% of the total pDNA.

The term “separation matrix” is used herein to denote a material comprising a support material to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support material. The terms “linker” and “extender” may be used to describe such a compound. The term “resin” is sometimes used for a separation matrix in this field. The terms “chromatography material” and “chromatography matrix” are used herein to denote a type of separation matrix.

Generally, a separation matrix may be contained in any type of separation device. The term “separation device” has its conventional meaning in the field of bioprocessing and is to be understood as encompassing any type of separation device which is capable of and suitable for separating and purifying compounds from a fluid containing by-products from the production of the compounds. A separation device may comprise a separation matrix, as defined above. Non-limiting examples of separation devices include chromatography columns and membrane devices, as further described elsewhere herein. According to a non-limiting example, a chromatography material in the form of porous particles may be packed in a chromatography column, before adding a liquid sample to the chromatography material being contained in the chromatography column. According to another non-limiting example, a chromatography material in the form of a membranous structure may be contained in a membrane device.

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”. Herein, the molecules in a liquid sample which interact with a ligand are referred to as “analyte”. The analyte of interest according to the present disclosure is supercoiled pDNA. Consequently, herein the terms “analyte” and “supercoiled pDNA” may be used interchangeably.

The chromatography material applied in the herein disclosed methods comprises a support material to which the ligand is coupled.

As mentioned above, the support material of the first chromatography material is a support material allowing convective flow through the first chromatography material. The term “support material allowing convective flow” is intended to mean a support material in which application of a hydraulic pressure difference between the inflow and outflow of the support material forces perfusion of the support material, achieving substantially convective transport of substance(s) into the support material or out of the support material.

Non-limiting examples of support materials allowing convective flow are nanofibres, a monolith, a membranous structure, and particles to which extenders are coupled.

Where the support material of the first chromatography material comprises nanofibers, such nanofibers may for example comprise electrospun polymer nanofibers. When in use, such nanofibers form a stationary phase comprising a plurality of pores through which a mobile phase can permeate. Herein, the terms “monolith” and “monolithic structure” have their conventional meaning in the field of bioprocessing. A monolith may be described as being single-unit structure. A monolith used in bioprocessing applications has a sponge-like structure including pores. Where the support material of the first chromatography material comprises a monolithic structure, a person skilled in the art is able to choose a monolithic structure having pore sizes suitable for the purpose of separating pDNA from a liquid sample.

Non-limiting examples of monolithic support material of the first chromatography material are Convective Interaction Media (CIM™ monoliths (BIA Separations), such as CIMmultus™ DEAE (Sartorius AG).

The support material of the first chromatography material may comprise a membranous structure, such as a single membrane, a pile of membranes or a filter. The membrane may be an adsorptive membrane. Where the support material of the chromatography material comprises a membranous structure, the skilled person is able to select a membranous structure having pore sizes suitable for the purpose of separating pDNA from a liquid sample. Where the chromatography material comprises a membranous structure, such membranous structure may for example comprise a nonwoven web of polymer nanofibers.

Non-limiting examples of suitable polymers may be selected from polysulfones, polyethersulfone, polyamides, nylon, polyacrylic acid, polymethacrylic acid, polyacrylonitrile, polystyrene, and polyethylene oxide, and mixtures thereof.

Alternatively, the polymer may be a cellulosic polymer, such as selected from a group consisting of cellulose and a partial derivative of cellulose, particularly cellulose ester, cross-linked cellulose, grafted cellulose, or ligand-coupled cellulose. Cellulose fiber chromatography (known as Fibro chromatography; Cytiva, Sweden) is an ultrafast chromatography purification for short process times and high productivity, which utilizes the high flow rates and high capacities of cellulose fiber. A non-limiting example of a suitable membranous structure comprises a reinforced cellulose.

Another non-limiting example of a suitable membranous structure comprises nylon.

Another non-limiting example of a suitable membranous structure comprises polyethersulfone, e.g. Mustang™ Q (Pall Corporation).

A non-limiting example of the first chromatography material, applied in step (a) of the herein disclosed separation method, comprises an anion exchange chromatography ligand and a support material having a membranous structure comprising cellulose or a partial derivative of cellulose, as described in detail elsewhere herein.

Another non-limiting example of membranous support material of the first chromatography material is a Sartobind® Q membrane adsorber (Sartorius AG), which has a macro-porous structure with pore size of >3 μm. Quaternary ammonium ligands are bound covalently to the complete internal surface of the membrane.

The term “membrane chromatography” has its conventional meaning in the field of bioprocessing. In membrane chromatography there is binding of components of a fluid, for example individual molecules, associates or particles, to the surface of a solid phase in contact with the fluid. The active surface of the solid phase is accessible for molecules by convective transport. The advantage of membrane adsorbers over packed chromatography columns is their suitability for being run with much higher flow rates. This is also called convection-based chromatography. A convection-based chromatography matrix includes any matrix in which application of a hydraulic pressure difference between the inflow and outflow of the matrix forces perfusion of the matrix, achieving substantially convective transport of substance(s) into the matrix or out of the matrix, which is effected very rapidly at a high flow rate. Convection-based chromatography and membrane adsorbers are described in for example US20140296464A1, US20160288089A1, WO2018011600A1, WO2018037244A1, WO2013068741A1, WO2015052465A1, U.S. Pat. No. 7,867,784B2, hereby incorporated by reference in their entirety.

The support material of the first chromatography material may be in the form of particles, such as substantially spherical, elongated or irregularly formed particles.

Suitable particle sizes may be in a diameter range of 5-500 μm. In the case of substantially spherical particles, the average particle size may be in the range of 5-1000 μm. The skilled person is able to choose the suitable particle size and porosity depending on the process to be used. For example, for a large-scale process, for economic reasons, a more porous but rigid support may be preferred to allow processing of large volumes, especially for the capture step. In chromatography, process parameters such as the size and the shape of the column will affect the choice. In an expanded bed process, the matrix commonly contains high density fillers, preferably stainless-steel fillers. For other processes other criteria may affect the nature of the matrix.

The support material may be made from an organic or inorganic material and may be porous or non-porous. In one embodiment, the support material is prepared from a native polymer, such as cross-linked carbohydrate material, e.g. agarose, agar, cellulose, dextran, chitosan, konjac, carrageenan, gellan, alginate, pectin, starch, etc. The native polymer support materials are easily prepared and optionally cross-linked according to standard methods, such as inverse suspension gelation (S Hjerten: Biochim Biophys Acta 79 (2), 393-398 (1964). In an especially advantageous embodiment, the support material is a kind of relatively rigid but porous agarose, which is prepared by a method that enhances its flow properties, see e.g. U.S. Pat. No. 6,602,990 (Berg). In an alternative embodiment, the support material is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g. “Styrene based polymer supports developed by suspension polymerization” (R Arshady: Chimica e L′Industria 70(9), 70-75 (1988)). Native or synthetic polymer support materials are also available from commercial sources, such as Cytiva, Sweden, for example in the form of porous particles. In yet an alternative embodiment, the support material is prepared from an inorganic polymer, such as silica. Inorganic porous and non-porous supports are well known in this field and easily prepared according to standard methods.

In the herein disclosed method for separating supercoiled pDNA, where the first chromatography material comprises a support material in the form of particles, the ligand is coupled to the support material via a longer linker molecule, also known as an extender. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo-or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl-and polymethacrylamides, polyvinyl ethers etc. Typical biopolymers are polysaccharides, such as starch, cellulose, dextran, agarose.

A non-limiting example of a particle-based support material of the first chromatography material is Fractogel™ (Merck Millipore), consisting of synthetic methacrylate based polymeric beads having tentacles of covalently attached linear polymer chains with functional ligands.

The anion exchange chromatography ligand of the first chromatography material may be a strong anion exchange chromatography ligand. The strong anion exchange chromatography ligand may for example comprise a quaternary amine group or a quaternary amino ethyl group.

Alternatively, the anion exchange chromatography ligand of the first chromatography material may be a weak anion exchange chromatography ligand. The weak anion exchange chromatography ligand may for example comprise a diethylaminoethyl (DEAE) group, a trimethylaminoethyl (TMAE) group, a dimethylaminoethyl (DMAE) group, or a trimethylhydroxypropyl (QA) group.

The first chromatography material used in the Example herein is Mustang™ Q (Pall Corporation), which comprises a support material in the form of a membranous structure, allowing convective flow through the first chromatography material, and a strong anion exchange ligand having a quaternary amine group, which ligand is covalently attached to the support material.

The second chromatography material comprises a support material to which the ligand is bound, which support material comprises nanofibres, a monolith, a membranous structure, or particles.

The detailed description of nanofibres, monoliths, membranous structures and particles found above in relation to support material of the first chromatography material is equally applicable to nanofibres, monoliths, membranous structures and particles in relation to support material of the second chromatography material.

However, a difference compared to the support material of the first chromatography material is that the support material of the second chromatography material does not necessarily have to be a support material allowing convective flow.

Accordingly, where the support material of the second chromatography material used are particles, the particles do not have to comprise an extender, but they may optionally comprise a linker connecting the ligand to the support, i.e., the coupling of the ligand to the support material may be provided by introducing a linker between the support material and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person. Optionally, the linker is an extender, as described in detail further above.

As mentioned further above, the second chromatography material comprises a ligand that binds to pDNA and enables selective separation of supercoiled pDNA from open circular pDNA. Selective separation may be achieved by the ligand of the second chromatography material either by being capable of increased binding interaction with supercoiled pDNA compared to its binding interaction with open circular pDNA (meaning that supercoiled pDNA binds stronger than open circular pDNA to the ligand of the second chromatography material), or by being capable of increased binding interaction with open circular pDNA compared to its binding interaction with supercoiled pDNA (meaning that open circular pDNA binds stronger than supercoiled pDNA to the ligand of the second chromatography material).

The ligand of the second chromatography material may enable salt-promoted affinity binding and/or thiophilic aromatic adsorption binding, to pDNA.

The ligand of the second chromatography material may for example comprise a thiophilic group, preferably a 2-mercaptopyridine group.

The second chromatography material used in the Example herein is Capto™ PlasmidSelect (Cytiva, Sweden), which comprises a support material in the form of porous agarose particles (median particle size of the cumulative volume distribution (d50V) is 36-44 μm), and a thiophilic aromatic adsorption ligand comprising a 2-mercaptopyridine group. This ligand is capable of increased binding interaction with supercoiled pDNA compared to its binding interaction with open circular pDNA.

Another non-limiting example of a second chromatography material is PlasmidSelect Xtra (Cytiva, Sweden), which comprises a support material in the form of porous agarose particles (median particle size of the cumulative volume distribution (d50V) is approx. 34 μm), and a thiophilic aromatic adsorption ligand comprising a 2-mercaptopyridine group.

The term “surface” herein means all external surfaces and includes in the case of a porous support material outer surfaces as well as pore surfaces.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a separation matrix after having added the liquid sample onto the separation matrix.

As mentioned above, in the method for separating supercoiled pDNA, the liquid sample originates from a cell culture harvest.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture may comprise intact cells, disrupted cells, a cell homogenate, and/or a cell lysate.

The term “cell culture harvest” is used herein to denote a cell culture which has been harvested and removed from the vessel or equipment, in which the cells have been cultivated.

As mentioned above, the liquid sample, added to the first chromatography material in step (a) of the above-described separation method, originates from a cell culture harvest and has been subjected to a step of removing RNA before step (a).

The skilled person understands that a liquid sample originating from a cell culture harvest contains many different types of biological compounds, including RNA, which need to be removed at some point of the purification process.

Herein, the term “removing RNA” is intended to mean reducing the amount of RNA present in the liquid sample. In the context of the presently disclosed method, the step of removing RNA is done before step (a), since RNA would compete with pDNA in the capture step, i.e., in binding to the first chromatography material. Thus, it is an advantage to remove RNA before step (a), to improve pDNA binding capacity of the first chromatography material applied in step (a). Preferably, the removal of RNA before step (a) results in a significant reduction of the amount of RNA in the liquid sample.

The liquid sample added to the first chromatography material in step (a) may thus be a pre-treated liquid sample.

Accordingly, the above-described method may comprise a step (a1) preceding step (a) as shown in FIG. 2, wherein step (a1) comprises pre-treating the liquid sample, optionally wherein said pre-treating comprises subjecting the cell culture harvest to one or more of cell lysis, precipitation of RNA in the presence of an RNA-precipitating agent, clarification, and filtration. Preferably said pre-treating comprises at least cell lysis, precipitation of RNA in the presence of an RNA-precipitating agent, and clarification. Precipitated RNA may be removed by filtration.

Different techniques for performing cell lysis are well known in the art. Commonly, harvested cells are mixed with an alkaline lysis solution to form a composition of lysed cells, also referred to as cell lysate. The lysis is subsequently interrupted by addition of a neutralization solution to the cell lysate, and cell debris, including fragmented cells, host cell proteins and host cell genomic DNA may then be removed.

For example, a cell culture may be subjected to alkaline lysis, neutralisation and/or clarification as described in International Patent Application No. PCT/EP2022/077388. For example, a cell culture may be contacted with an alkaline lysis solution in a single use vessel, such as a single use bag, for alkaline lysis of said cells, wherein the single use vessel comprises an integrated mixer, and the cell culture is mixed with the alkaline lysis solution. The lysis may be interrupted by addition of a neutralization solution to the cell culture.

Alternatively, harvested cells present in a liquid sample (e.g. in the form of a slurry) may be mixed with the alkaline lysis solution by means of a static mixer. Static mixers as such are known. A static mixer useful in the present context may be an elongated tubular member containing a plurality of baffles arranged within the interior of the tubular member such as to partially obstruct the flow path of a fluid passing through the tubular member. A static mixer thus achieves a turbid flow that provides mixing of the components of the fluid passing therethrough. The baffles of the static mixer may have a rounded shape. With such shape, the flow of fluid containing cell lysate and free plasmids is gently redirected, avoiding damage to the plasmids. To mix a slurry of harvested cells with alkaline lysis solution, the alkaline lysis solution may be added via a fluid inlet to a vessel or tube containing the cell slurry, and the combined composition may then be forwarded (e.g., pumped) to and through the static mixer. For example, the cell slurry may be provided via one inlet to a joint, and the alkaline lysis solution may be provided via another inlet to said joint. The joint may be a Y joint. The mixture may be pumped continuously and at constant flow rate through the static mixer.

After being mixed, optionally using a static mixer, the alkaline lysis solution remains in contact with the cell slurry for a time sufficient for cell lysis to proceed to a sufficient degree, preferably completely. The contact time may be in the range of from 1 minute and up to 10 minutes, and may be between 2 and 5 minutes, such as from 2 to 3 minutes. The longer the contact time, the higher the risk that some of the plasmid DNA will also degrade. Hence, a contact times of less than 10minutes is preferred.

The lysis reaction is interrupted by mixing the cell lysate obtained with a neutralization solution as described above.

The addition of the neutralization solution may occur in a single use vessel which may be the same single use vessel as used during alkaline lysis, or it may be a separate single use vessel, which is the case where a static mixer is used as described above. The single use vessel within which the cell lysate is contacted with the neutralization solution may comprise an integrated mixer.

Where a static mixer is used for mixing the cell slurry and the alkaline lysis solution, the neutralization solution may be added to the cell lysate via a fluid inlet arranged after (downstream of) the static mixer, and the neutralized cell lysate may then be transferred to a vessel where further processing, such as RNA precipitation, clarification and/or filtration, may occur. Following lysis and neutralization, the cell lysate may optionally be subjected to RNA precipitation using an RNA precipitating agent. The RNA-precipitating agent may be selected from the group consisting of calcium chloride (CaCl₂), lithium chloride (LiCl), and tripotassium citrate (C₆H₅K₃O₇); preferably calcium chloride.

Following lysis and neutralization, the cell lysate may be subjected to clarification and/or filtration. The skilled person has knowledge of various methods for clarification and methods for filtration of liquid samples originating from a cell culture harvest. For instance, the cell lysate may be clarified essentially as described in PCT/EP2022/077388, by
after addition of neutralization solution to interrupt the lysis, optionally transferring the cell lysate to a single use vessel, such as a single use mixing bag,
clarifying the cell lysate in a single use vessel by adding a flocculate lifting agent into the single use vessel, whereby at least two phases are separated inside the single use vessel, whereby one of said phases is a neutralized and clarified alkaline solution comprising plasmid DNA;
removing the neutralized and clarified alkaline solution from the single use vessel;
filtering the neutralized and clarified alkaline solution.

The single use vessel in which clarification is performed may contain an integrated mixer. The single use vessel of the clarification step may be a single use bag, such as a flexible plastic bag.

One or more of the process steps described herein may be performed as part of a continuous process, preferably using single use equipment such as single use bags, tubing and mixer(s). For example, cell lysis using a static mixer as described above, followed by neutralization may be performed in a continuous process. Advantageously such a process can be readily controlled by adjusting the settings of one or more pumps controlling the flow rate of cell harvest slurry, lysis solution and neutralization solution.

If not continuous, steps are performed batch-wise. For example, cell lysis and neutralization may be performed continuously, followed by batch RNA precipitation, clarification and filtration. However, it is also conceivable that all steps may be performed batchwise. Furthermore, a process for purification of plasmid DNA involving cell lysis using a static mixer as described above, followed by neutralization, RNA precipitation, clarification and filtration, whether performed continuously or in batch, may be followed by the chromatography steps as described herein, or other chromatography and/or filtration techniques.

As mentioned above, the supercoiled pDNA eluted in step (d) has a purity degree of at least 95%, such as 96%, 97%, 98%, 99%, or 99.5%, without use of any further chromatography material than said first and second chromatography materials. Accordingly, the presently disclosed method for separating supercoiled pDNA achieves an excellent degree of purification by employing only two chromatography steps. No additional chromatography step is needed to obtain supercoiled pDNA having a purity degree of at least 95%.

The above-described method may comprise a step (c1) between step (b) and step (c), wherein step (c1) comprises diluting the liquid sample eluted in step (b) in a diluting agent at a volume ratio of at least 1:2, such as 1:3, 1:4 or 1:5, of liquid sample to diluting agent. The diluting agent comprises a salt. Currently preferred salt is ammonium sulfate ((NH₄)₂SO₄). Currently preferred volume ratio is 1:4 of liquid sample to ammonium sulfate. Currently preferred concentration of ammonium sulfate is 3M. It is contemplated that other salts with similar properties as ammonium sulfate according to the

Hofmeister series may be suitable to use instead of ammonium sulfate. The Hofmeister series is a classification of ions in order of their ability to salt out or salt in protein. The diluting agent may optionally act as a buffering agent.

The above-described method may comprise a step (d1) between step (c) and step (d), wherein step (d1) comprises eluting the open circular pDNA from the second chromatography material. Under some processing conditions, the open circular pDNA will bind to the second chromatography material, in which case it needs to be eluted from the second chromatography material. As explained further above, the second chromatography material enables selective separation of supercoiled pDNA from open circular pDNA. Where the second chromatography material is capable of increased binding interaction with supercoiled pDNA compared to its binding interaction with open circular pDNA, supercoiled pDNA binds stronger than open circular pDNA to the second chromatography material. Thereby, the open circular pDNA will be easier to elute from the second chromatography material than supercoiled pDNA. In this case, step (d1) is performed before step (d).

As mentioned further above, one of the advantages of the herein disclosed method is a significant reduction in production time of downstream processing. In fact, the herein disclosed method for separating supercoiled pDNA has been optimized such that steps (a)-(d) and any intermediate steps can be completed within 5 hours. This is a result of a combination of features described above. More particularly, the combination of having subjected the liquid sample to a step of removing RNA before step (a), a first chromatography material comprising a support material allowing convective flow, and a second chromatography material comprising a ligand that enables selective separation of supercoiled pDNA from open circular pDNA, makes it possible to complete the above-described steps (a), (b), (c), and (d), and any intermediate steps, such as the above-described steps (c1) and (d1), within 5 hours.

The above-mentioned time frame “within 5 hours” required for steps (a)-(d) and any intermediate steps, does not include any step performed before step (a), such as the above-described step (a1), and does not include any step performed after step (d), such as the below-described steps (e1), (e2), (e3).

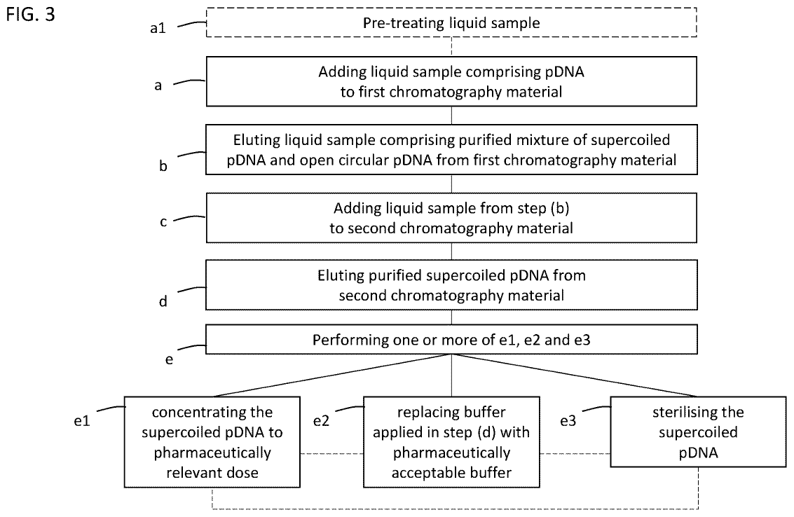

The herein disclosed method for separating supercoiled pDNA may further comprise subjecting the supercoiled pDNA eluted in step (d) to one or more of the following steps, as shown in FIG. 3:
(e1) concentrating the supercoiled pDNA to a pharmaceutically relevant dose,
(e2) replacing a buffer applied in step (d) of the above-described method with a pharmaceutically acceptable buffer, and/or
(e3) sterilizing the supercoiled pDNA,
   thereby obtaining a composition comprising pharmaceutical grade supercoiled pDNA, optionally wherein the composition is a pharmaceutical composition or an active pharmaceutical ingredient comprising pharmaceutical grade supercoiled pDNA.

A person skilled in the art understands that the pharmaceutically relevant dose will depend on various factors such as, but not limited to, the disease or disorder to be treated as well as the weight and condition of the subject to be treated with a pharmaceutical composition or an active pharmaceutical ingredient.

Pharmaceutically acceptable buffers are well known in the art and can easily be chosen by the skilled person.

For the resulting composition to fulfil all regulatory requirements for pharmaceutical compositions and active pharmaceutical ingredients, normally all of the above-listed three steps e1-e3 have to be performed.

The present disclosure further provides a method for preventing or treating a disease or disorder in a subject, comprising administering to the subject a pharmaceutical composition or an active pharmaceutical ingredient comprising pharmaceutical grade supercoiled pDNA obtained by performing the above-disclosed separation method comprising one or more of steps e1-e3 (as described in detail above).

The active pharmaceutical ingredient may be administered in the form of a DNA vaccine, and the disease or disorder to be prevented is caused by an infection by a virus, such as SARS-COV-2, Zika, Influenza A, yellow fever virus, or human immunodeficiency virus (HIV).

The present disclosure also provides a method for producing a viral vector, comprising transfecting cells in vitro with a composition comprising pharmaceutical grade supercoiled pDNA obtained by performing the above-disclosed separation method comprising one or more of steps e1-e3, thereby enabling production of a viral vector.

The viral vector may be for use as an active pharmaceutical ingredient. It is to be understood that said viral vector may comprise therapeutically relevant genetic material originating from the pharmaceutical grade supercoiled pDNA. The active pharmaceutical ingredient may be in the form of a viral vector vaccine for use in a method of preventing a disease or disorder caused by an infection by a virus. The active pharmaceutical ingredient may be for use in a method of cell therapy, gene therapy or oncolytic therapy. Oncolytic therapy may comprise treatment of a cancer, such as breast cancer, lung cancer, ovarian cancer or leukemia. The active pharmaceutical ingredient may be for use in a method for treating or preventing a disease or disorder such as cystic fibrosis, Leber's congenital amaurosis, or spinal muscular atrophy.

The present disclosure further provides a method for producing RNA, comprising using the composition comprising pharmaceutical grade supercoiled pDNA obtained by performing the above-disclosed separation method comprising one or more of steps e1-e3, as a template to produce RNA.

Optionally, the RNA thus produced may be for use as an active pharmaceutical ingredient. It is to be understood that said RNA may comprise therapeutically relevant genetic material originating from the pharmaceutical grade supercoiled pDNA. The active pharmaceutical ingredient may be in the form of an RNA vaccine for use in a method of preventing a disease or disorder caused by an infection by a virus, such as SARS-COV-2, Zika, Influenza A, human immunodeficiency virus (HIV), or rabies virus. The active pharmaceutical ingredient may be for use in a method of gene therapy, oncolytic therapy, or personalized medicine, or for treating or preventing a disease or disorder, such as a rare disease, cystic fibrosis, Leber's congenital amaurosis, or spinal muscular atrophy. Oncolytic therapy may include treatment of a cancer such as breast cancer, lung cancer, ovarian cancer or leukemia.

A person of skill in the art understands that the pharmaceutical composition or active pharmaceutical ingredient must be administered in a pharmaceutically effective amount or dose to the subject to achieve the desired medical effects. Amounts and doses which are pharmaceutically effective depend on various factors such as, but not limited to, the disease or disorder to be treated or prevented, as well as the weight and condition of the subject to be treated.

Devices or compositions “comprising” one or more recited components may also include other components not specifically recited. The term “comprising” includes as a subset “consisting essentially of” which means that the device or composition has the components listed without other features or components being present. Likewise, methods “comprising” one or more recited steps may also include other steps not specifically recited.

The singular “a” and “an” shall be construed as including also the plural.

### Example: Separation of Supercoiled pDNA from cell culture harvest

### Methods and Materials

E. coli cells (clone DH5α) comprising plasmid DNA (pDNA) having a size of 7.3 kbp were harvested by centrifugation of a fermentation liquid from a fed-batch process in a 15 L stirred-tank bioreactor. The fermentation conditions included pH=7.2±0.2, dissolved oxygen (DO)=30%, and temp.=37° C. The feed profile supported a specific growth rate of 0.25 h-1. Targeted final OD was 45 to 50. The process was terminated after 16.5 h.

The E. coli cell culture harvest thereby obtained was subjected to alkaline lysis to disrupt the cells, followed by neutralisation, i.e., adjusting the pH to about 5, of the resulting liquid sample.

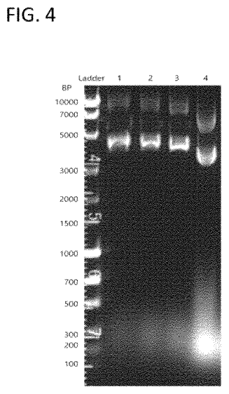

Thereafter, RNA was precipitated by addition of CaCl₂ to said liquid sample. Since RNA would compete with pDNA in binding to the chromatography material in the subsequent capturing chromatography step, it is advantageous to remove RNA before the capturing step to improve pDNA binding capacity of the chromatography material. FIG. 4 shows that by adding CaCl₂ as RNA precipitating agent, the amount of RNA in the liquid sample is significantly reduced. FIG. 4: lane (1) 1M CaCl₂, lane (2) 0.75 M CaCl₂, lane (3) 0.5 M CaCl₂, and lane (4) no CaCl₂ added).

Subsequently, the liquid sample was clarified by subjecting it to a flocculation lift step, followed by a filtration step, thereby removing RNA precipitate and flocculate from the liquid sample. Conditions: Depth filtration (PDP8, Pall Corporation): Flow rate 100 LMH (L/m²/h), load 220 L/m² Ultrafiltration/Diafiltration (UF/DF): Shear rate 6000 s⁻¹, transmembrane pressure (TMP) 0.07 MPa (0.7 bar, 10.2 psi), load 70 L/m²

Normal flow filtration (NFF) (NFF filter, Pall Corporation): Flow rate 600 LMH (L/m²/h), load 180 L/m²

The resulting pre-treated liquid sample was then subjected to two chromatography steps, described in detail below, thereby obtaining supercoiled pDNA at a purity degree of 95% or higher.

### First Chromatography Step

Capturing of pDNA, i.e., separation of pDNA from the pre-treated liquid sample, by use of a first chromatography material comprising an anion exchange chromatography ligand which is capable of binding to pDNA.

First chromatography material: Mustang™ Q membrane (Pall Corporation), having a ligand comprises a quaternary amine group.

A Mustang™ Q XT5 (5 mL) membrane capsule was used.

Separation conditions:

Three cycles were performed on the Mustang™ Q membrane, with a load of liquid sample of 150 mL (cycle 1), 200 ml (cycle 2), and 200 ml (cycle 3), respectively.

Load 12 mg pDNA/membrane volume (MV)

Binding at 500 mM NaCl, 50 mM Tris, pH 8.0

Elution at 3M NaCl, 50 mM Tris, pH 8.0

Flow rate 5 MV/min

For increase of yield, elution is paused for 30 min at UV increase

Cleaning-in-place (CIP) 1M NaOH

Regeneration 1M NaCl, 0.1M NaOH

### Second Chromatography Step

Separation of supercoiled pDNA from open circular pDNA by use of a second chromatography material comprising a ligand that binds to pDNA and enables selective separation of supercoiled pDNA from open circular pDNA.

Second chromatography material: Capto™ PlasmidSelect resin (Cytiva, Sweden), having a ligand comprises a 2-mercaptopyridine group.

The resin was packed in a HiScale™ 16 (20 mL) column, 10 cm bed height.

Separation conditions:

Load 2 mg pDNA/mL resin

Eluate dilution 1+4 in 3 M (NH₄)₂SO₄

Binding at 2.25 M (NH₄)₂SO₄

Flow rate: Equilibrium and load 220 cm/h; Elution 120 cm/h

Elution by linear gradient against water, 11 column volumes (CV), 55%

### Results and Conclusions

First chromatography step

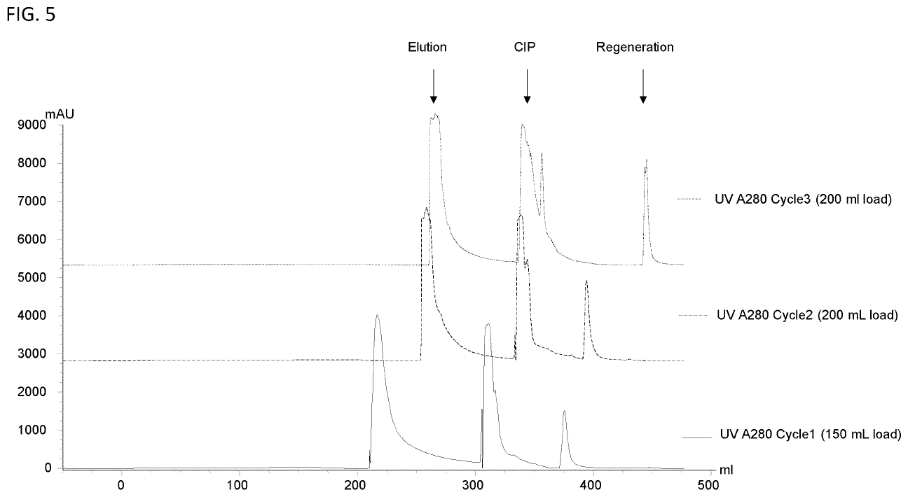

FIG. 5 shows reproducible and rapid cycling, as well as consistency at different load, on Mustang Q™ membrane. In the chromatogram of FIG. 5, the solid line at the bottom represents Cycle 1 (150 ml sample), the dashed line in the middle represents Cycle 2 (200 ml sample), and the dotted line on top represents Cycle 3 (200 mL sample). The peak to the left represents elution of pDNA (a mixture of supercoiled pDNA and open circular pDNA), the peak in the middle represents CIP, and the peak to the right represents regeneration of the membrane.

### Second Chromatography Step

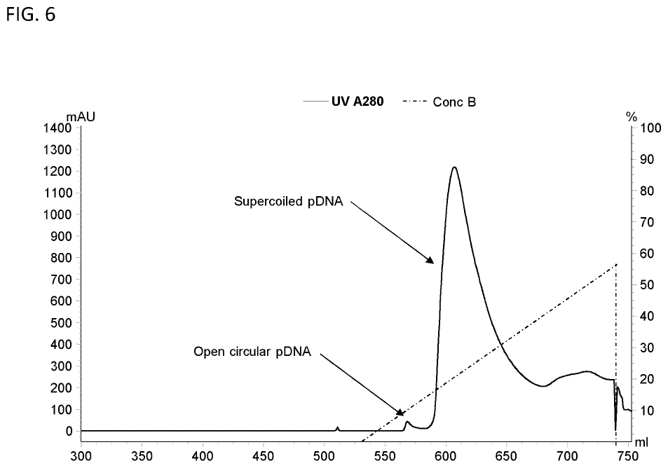

FIG. 6 shows successful purification of supercoiled pDNA, separated from open circular pDNA, using Capto™ PlasmidSelect resin. In the chromatogram of FIG. 6, it can be seen that open circular pDNA is eluted first (the small peak to the left), followed by elution of supercoiled pDNA (the large peak). Impurities such as RNA and endotoxins are eluted at the end of the gradient after the supercoiled pDNA. The impurity peak also contains step yield loss of pDNA. The dotted line named “Conc B” shows the salt gradient of elution buffer against pure water (decreasing salt concentration). After a final ultrafiltration/diafiltration, the purity degree of the supercoiled pDNA obtained was remarkably high (99.3%). Agarose gel electrophoresis (AGE) and capillary gel electrophoresis (CGE) confirmed the successful separation of supercoiled pDNA (not shown).

Table 1 shows the purity degree of supercoiled (SC) pDNA and content of open circular (OC) pDNA after each step of the separation method described in this Example.

TABLE 1
Content of supercoiled pDNA and open circular
pDNA throughout the separation process.

| Step in process | SC (%) | OC (%) |
|---|---|---|
| First UF/DF | 97.4 | 2.6 |
| Normal flow filtration (NFF) | 97.7 | 2.3 |
| Mustang ™ Q eluate | 92.1 | 7.9 |
| Capto ™ PlasmidSelect eluate | 97.4 | 2.6 |
| Final UF/DF retentate | 99.3 | 0.7 |

Table 2 shows calculations of the ammonium sulfate buffer consumption for a 200 L scale process in accordance with the above example (“New process”), and compared to the process disclosed in Application Note 28-4094-85 AA (Cytiva) (“prior art process”).

TABLE 2
Volume (L) of ammonium sulfate buffer consumed respectively in a prior
art process and the new process per 200 L cell culture harvest

| | | Prior art process | | New process | |
|---|---|---|---|---|---|
| | | Group | | 1^{st} chroma- | 2^{nd} chroma- |
| | | separation | Capture | tography step | tography step |
| Detailed | Equilibration | 96 L | 30 L | 0 | 30 L |
| process | Wash | 0 | 30 L | 0 | 20 L |
| step | Elution | 64 L | 20 L | 0 | 80 L |
| | Dilution* | — | — | 200 L | — |
| | Total per cycle | 160 L | 80 L | 200 L | 130 L |

| No. of cycles | 5 | 2 | 1 | 2 |
|---|---|---|---|---|
| Volume | 800 L | 160 L | 200 L | 260 L |
| Total volume per 200 L | 960 L | | 460 L | |
| cell culture harvest | | | | |

*Dilution is performed between the first and second chromatography steps of the method disclosed herein, but can also be referred to as part of the second chromatography step. However, the sample is diluted only once, even if the following 2^{nd} chromatography step is repeated.

Compared to a previously known method for purification of supercoiled pDNA, including three chromatography steps (Application Note 28-4094-85 AA, Cytiva, Sweden), an 80% reduction in production time for downstream processing, and a 50% reduction in ammonium sulfate consumption was achieved.

It is to be understood that the present disclosure is not restricted to the above-described exemplifying embodiments thereof and that several conceivable modifications of the present disclosure are possible within the scope of the appended claims.

### REFERENCES

Lemmens R et al., Supercoiled plasmid DNA: selective purification by thiophilic/aromatic adsorption, Journal of Chromatography B, 5 Feb. 2003, 784 (2):291-300

Sandberg L M et al., Thiophilic interaction chromatography for supercoiled plasmid DNA purification, Journal of Biotechnology, 8 Apr. 2004, 109 (1-2):193-199Application Note 28-4094-85 AA, Cytiva, Sweden

Hitchcock A G et al., Scale-up of a plasmid DNA purification process, Use of a commercial resin to produce GMP-grade pDNA for clinical studies, BioProcess International, December 2010, 8 (11): 46-54

1. A method for separating supercoiled plasmid DNA (pDNA) from a liquid sample, the method comprising the steps of:
(a) adding a liquid sample comprising pDNA to a first chromatography material comprising (i) an anion exchange chromatography ligand for binding to pDNA and (ii) a support material allowing convective flow through the first chromatography material, wherein the liquid sample originates from a cell culture harvest and has been subjected to a step of removing RNA before step (a);
(b) eluting a liquid sample, comprising a purified mixture of supercoiled pDNA and open circular pDNA, from the first chromatography material;
(c) adding the liquid sample from step (b) to a second chromatography material comprising a ligand that binds to pDNA and enables selective separation of supercoiled pDNA from open circular pDNA;
(d) eluting the purified supercoiled pDNA from the second chromatography material;
wherein the supercoiled pDNA eluted in step (d) has a purity degree of at least 95% without use of any further chromatography material than said first and second chromatography materials.

2. The method of claim 1, wherein the anion exchange chromatography ligand is a strong anion exchange chromatography ligand, optionally wherein the strong anion exchange chromatography ligand comprises a quaternary amine group or a quaternary amino ethyl group.

3. The method of claim 1, wherein the anion exchange chromatography ligand is a weak anion exchange chromatography ligand, optionally wherein the weak anion exchange chromatography ligand comprises a diethylaminoethyl (DEAE) group, a trimethylaminoethyl (TMAE) group, a dimethylaminoethyl (DMAE) group, or a trimethylhydroxypropyl (QA) group.

4. The method of claim 1, wherein the ligand of the second chromatography material enables salt-promoted affinity binding, and/or thiophilic aromatic adsorption binding, to pDNA.

5. The method of claim 4, wherein the ligand of the second chromatography material comprises a thiophilic group.

6. The method of claim 5, wherein the ligand of the second chromatography material comprises a 2-mercaptopyridine group.

7. The method of claim 1, wherein the support material of the first chromatography material comprises a material selected from the group consisting of nanofibres, a monolith, a membranous structure, and particles to which extenders are coupled.

8. The method of claim 7, wherein the support material of the first chromatography material comprises electrospun polymer nanofibers.

9. The method of claim 7, wherein the support material of the first chromatography material comprises membranous structure comprising a nonwoven web of polymer nanofibers.

10. The method of claim 7, wherein the support material of the first chromatography material comprises particles to which extenders are coupled, wherein the particles are substantially spherical particles.

11. The method of claim 1, wherein the second chromatography material comprises a support material to which the ligand is bound, which support material comprises a material selected from the group consisting of nanofibres, a monolith, a membranous structure, and particles.

12. The method of claim 11, wherein the support material of the second chromatography material comprises particles, wherein the particles are substantially spherical particles.

13. The method of claim 11, wherein the support material of the second chromatography material comprises electrospun polymer nanofibers.

14. The method of claim 11, wherein the support material of the second chromatography material comprises a membranous structure comprising a nonwoven web of polymer nanofibers.

15. The method of claim 1, further comprising a step (a1) preceding step (a), wherein step (a1) comprises pre-treating the liquid sample, wherein said pre-treating comprises at least one of: subjecting the cell culture harvest to cell lysis, precipitation of RNA in the presence of an RNA-precipitating agent, clarification, and filtration.

16. The method of claim 15, wherein step (a1) comprises
(a1-a) subjecting the cell culture harvest to cell lysis;
(a1-b) precipitation of RNA in the presence of an RNA-precipitating agent; and
(a1-c) clarification.

17. The method of claim 15, wherein said pre-treating comprises subjecting the cell culture harvest to cell lysis by mixing the cell culture harvest with a lysis solution using a static mixer.

18. The method of claim 17, wherein the cell culture harvest is contacted with said lysis solution to form a combined composition, and said combined composition is allowed to flow through said static mixer to achieve mixing thereof.

19. The method of claim 15, wherein said pre-treating comprises precipitation of RNA in the presence of an RNA-precipitating agent, and the RNA-precipitating agent is selected from the group consisting of calcium chloride, lithium chloride, and tripotassium citrate; preferably calcium chloride.

20. A method for producing a purified composition comprising pDNA, comprising
(I) providing a cell culture harvest comprising pDNA producer cells;
(II) contacting said cell culture harvest with a lysis solution to form a lysis composition;
(III) mixing the lysis compositing using a static mixer to form a cell lysate;
(IV) adding a neutralization solution to the cell lysate;
(V) optionally precipitating RNA in the cell lysate by action of an RNA-precipitating agent;
(VI) clarifying the cell lysate;
(VII) filtering the cell lysate to obtain a solution comprising plasmid DNA (pDNA)
(VIII) subjecting said solution comprising pDNA to at least one chromatographic purification step to provide a purified composition comprising pDNA.

21. The method of claim 20, wherein said at least one chromatographic purification step comprises at least one bind-elute chromatography step to capture pDNA.

22. The method of claim 20, wherein said at least one chromatographic purification step comprises adding the solution comprising pDNA a chromatography material comprising a ligand that binds to pDNA and enables selective separation of supercoiled pDNA from open circular pDNA, and eluting the purified supercoiled pDNA from the second chromatography material.

23. The method of claim 20, wherein the static mixer is an inline static mixer.

24. The method of claim 24, wherein steps (I) to (VIII) are performed continuously.

25. The method of claim 20, wherein step (V) is performed, and step (VIII) comprises
(a) adding the solution comprising pDNA to a first chromatography material comprising (i) an anion exchange chromatography ligand for binding to pDNA and (ii) a support material allowing convective flow through the first chromatography material;
(b) eluting a liquid sample, comprising a purified mixture of supercoiled pDNA and open circular pDNA, from the first chromatography material;
(c) adding the liquid sample from step (VIII-b) to a second chromatography material comprising a ligand that binds to pDNA and enables selective separation of supercoiled pDNA from open circular pDNA;
(d) eluting the purified supercoiled pDNA from the second chromatography material;
wherein the supercoiled pDNA eluted in step (d) has a purity degree of at least 95% without use of any further chromatography material than said first and second chromatography materials.

26. The method of claim 25, wherein the first chromatography material and/or the second chromatography material is as defined.

27. The method of claim 1, further comprising a step (c1) between step (b) and step (c), wherein step (c1) comprises diluting the liquid sample eluted in step (b) in a diluting agent at a volume ratio of at least 1:2, such as 1:3, 1:4 or 1:5, of liquid sample to diluting agent, wherein the diluting agent comprises a salt.

28. The method of claim 27, wherein the diluting agent comprises ammonium sulfate.

29. The method of claim 1, further comprising a step (d1) between step (c) and step (d), wherein step (d1) comprises eluting the open circular pDNA from the second chromatography material.

30. The method of claim 1, further comprising subjecting the supercoiled pDNA eluted in step (d) to one or more of the following steps:
(e1) concentrating the supercoiled pDNA to a pharmaceutically relevant dose,
(e2) replacing a buffer applied in step (d) of claim 1 with a pharmaceutically acceptable buffer, and/or
(e3) sterilizing the supercoiled pDNA, thereby obtaining a composition comprising pharmaceutical grade supercoiled pDNA, optionally wherein the composition is a pharmaceutical composition or an active pharmaceutical ingredient comprising pharmaceutical grade supercoiled pDNA.

31. A method for producing a viral vector, comprising transfecting cells in vitro with a composition comprising pharmaceutical grade supercoiled pDNA obtained by performing the method of claim 30, thereby enabling production of a viral vector.

32. A method for producing RNA, comprising using the composition comprising pharmaceutical grade supercoiled pDNA obtained by performing the method of

30. as a template to produce RNA, optionally wherein the RNA is for use as an active pharmaceutical ingredient.

33. A method for preventing or treating a disease or disorder in a subject, comprising administering to the subject a pharmaceutical composition or an active pharmaceutical ingredient comprising pharmaceutical grade supercoiled pDNA obtained by performing the method of claim 30, optionally wherein the active pharmaceutical ingredient is administered in the form of a DNA vaccine, and the disease or disorder to be prevented is caused by an infection by a virus, such as SARS-COV-2, Zika, Influenza A, yellow fever virus, or human immunodeficiency virus (HIV).
